(19) United States
(12) Patent Application Publication
(10) Pub. No.: US 2020/0178833 A1
(43) Pub. Date: Jun. 11, 2020

(54) METHOD AND SYSTEM FOR MEASURING ELECTROPHYSIOLOGICAL SIGNALS WITH REAL TIME ADJUSTMENT OF SIZE, ELECTRODE POSITIONING, AND SPATIAL RESOLUTION OF A HEADSET

(71) Applicants: Saif Ullah, Islamabad (PK); Salman Hussain, Islamabad (PK); Mirza Jahanzaib, Taxila (PK); Wasim Ahmad, Wah Cantt (PK)

(72) Inventors: Saif Ullah, Islamabad (PK); Salman Hussain, Islamabad (PK); Mirza Jahanzaib, Taxila (PK); Wasim Ahmad, Wah Cantt (PK)

(21) Appl. No.: 16/793,037

(22) Filed: Feb. 18, 2020

(51) Int. Cl. A61B 5/0478; A61B 5/00

(52) U.S. Cl. CPC A61B 5/0478; A61B 2562/063; A61B 5/6831; A61B 5/6803

(57) ABSTRACT

A system for measuring electrophysiological signals from a human brain. The system includes a head set unit which has a adjustable strap attached to atop base and a circular base. A peripheral rack around the circular base allowing for strips containing electroencephalograph (EEG) sensors to be moved. Furthermore, the position of the EEG sensors may be changed on the straps and their height may be changed so that all EEG sensors are in contact with a human head.

### PRIORITY

This application claims the benefit of priority from Pakistan Application No. 104/2019, filed on Feb. 19, 2019, at the Intellectual Property Organization, Pakistan, and entitled “METHOD AND SYSTEM FOR MEASURING ELECTROPHYSIOLOGICAL SIGNALS WITH REAL TIME ADJUSTMENT OF SIZE, ELECTRODE POSITIONING, AND SPATIAL RESOLUTION OF THE HEADSET,” which is incorporated herein by reference in its entirety.

### TECHNICAL FIELD

This invention relates to a system and method to measure electrophysiological signals from the scalp of a subject. More specifically, the disclosure relates to a system and method comprising of headset assembly, processing machine and a wireless motor controller with a mechanism for real time adjustment of the length of dry Electroencephalograph (EEG) measuring electrode sensors according to the scalp size of the subject, positioning of dry EEG measuring electrode sensors on the scalp of subject and spatial resolution of the headset according to the user requirement in, real time.

### BACKGROUND

The present disclosure finds its genesis in measuring Electroencephalograph (EEG). As a background, a human brain contains more than 100 billion neurons and above 400 trillion neural connectors. A brain produces electrical signals as a result of an activity between the synapsis regions of the neurons where neurons makes connection with each other. EEG may be used to measure a strength of electrical signals which are transmitting between neurons due to an activity, in the form of brain waves. EEG measurements aid in brain function diagnosis, brain machine interphase, neural prosthetics and neuroimaging applications.

EEG measurements from the brain may be detected by placing electrodes on the scalp of a subject. The most common and conventional method for recording of EEG signals from a subject's scalp entails utilizing wet electrodes which are placed on the scalp of a subject based on a standard 10-20 system. A 10-20 system refers to a standard method used to describe and position the location of EEG measuring electrode sensors on the scalp of the subject. In a 10-20 system, “10” and “20” describes the actual distance between adjacent EEG electrode sensors as being either 10% or 20% of the total front-back or right-left distance of a skull.

Accurate placement of EEG measuring electrodes on the scalp of subject is critical and has significant importance for precise measurement of the EEG signals. Use of wet electrodes for EEG signals measurement require accurate marking where EEG measuring electrode sensor are placed on the scalp of the subject, require skin preparation and conducting gel is applied on the scalp. These things make EEG measurement time consuming, causes time varying offset in voltage signals of EEG measurement, irritating to the subject and needs cleaning of the head of subject after EEG measurement.

Numerous approaches have been developed to eliminate the limitations of wet electrodes EEG measurement using dry EEG electrodes.

In U.S. Ser. No. 00/615,4669A, a headset and system was developed to measure EEG signals using dry EEG electrodes. The dry EEG electrode sensors used in U.S. Ser. No. 00/615,4669A are formed of dry conductive foam rubber pads attached on each electrode.

In US20020183605A1, an array of dry EEG measuring electrode is constructed to allow the user to easily adjust to the correct size of the scalp of patient. The electrode array invented in US20020183605A1 is disposable, pre-gelled, self-preparing and uses multiple electrodes which can fit on most head sizes to detect cerebral functions.

In US20080225585A1, an EEG measuring headset comprising of multiple rigid bands with multiple number of predefined positioned voids to place dry EEG electrodes was developed. The invention uses chin strap to fix the headset with the head of the subject. However, the tightening processes of the strip may induce possibility to mismatch the position of EEG electrodes with the predefined position of EEG electrodes on the scalp of subject when the strip is tightened on the scalp. This can lead to detection of inaccurate EEG signals.

In US20110237923A1, a headset with plurality of dry EEG measuring electrodes is developed. A mechanism was disclosed which comprised adjusting the diameter of a headset, which may be fastened to the scalp of a subject.

In U.S. Pat. No. 9,215,978B2, a headset containing a central body portion and a plurality of extensions extending from the central body portion. Each extension contains a dry EEG measuring electrode and the device includes an adjustment band to adjust the position of the extensions on the scalp of the subject.

In US20170027466A1, a headset comprising of multiple number of cantilever arms which are bent outward for placement on the scalp of subject, is invented. The cantilever arms are used to place the dry EEG measuring electrode sensors and helps to grasp the scalp of the subject.

The conventional EEG headsets for EEG measurement use various fastening mechanisms to secure the direct contact of EEG electrodes with the scalp of the subject using straps and connecting devices (US 20110237923A1) or uses rigid bands (US20080225585A1) to use their headset on different size of scalp. The electrodes are placed on the predefined positions on the headset which may vary when they are tightly fastened on the subject head and may induce errors in the EEG measurements. In addition, in US20170027466A1, EEG electrodes can grasp the scalp of the subject which have possibility that when subject wear these headsets, the EEG sensors may not make contact with the scalp of the subject on the required area of scalp and ultimately cause errors in the EEG signal measurements. The prior headsets which have been developed are not flexible enough to adjust their size and the height of the EEG electrodes from the scalp of the subject to secure their predefined position and their direct contact with the scalp surface.

Conventional EEG measuring headsets have a limitation that they mostly provide predefined fixed positions for EEG measuring electrodes on headsets to measure the EEG signals. These headsets can be used when the EEG recordings from the scalp are only required on these fixed positions of the headsets. The prior headsets which have marked the position on headset to place EEG electrode sensors based on 10-20 system can only be used when the EEG signals from subject are required on the positions marked on 10-20 system. Accordingly, to measure calculations at other positions, a different headset is needed. Accordingly, conventional headsets do not allow enough flexibility for EEG measurements based on different positions of EEG electrodes on a single headset.

In addition, the prior art which invented EEG measuring headsets have fixed recording resolution and if the EEG measurements from the subject are needed with different recording resolution, these headsets are required to replace with the required recording resolution and it can increase the cost of operation and time. The prior headsets which have been developed are not flexible enough to measure the EEG measurements based on different resolutions from single headset.

As such, it would be beneficial to provide a system and method which may provide real time display of the EEG measurements of a subject from all EEG measuring electrode sensors along with the real time display and real time adjustment mechanism to modify respective heights of EEG electrode sensors from the surface of the scalp of subject, position of EEG measuring electrodes sensors on the scalp, and their spatial resolution on the headset.

### SUMMARY

An object of the invention is to provide new and improved methods and systems from a subject's head. The following presents a simplified summary of exemplary embodiments of the present disclosure in order to provide a basic understanding of some aspects described herein. This summary is not an extensive overview of the claimed subject matter. It is intended to neither identify key nor critical elements of the claimed subject matter nor delineate the scope of the subject innovation. Its sole purpose is to present some concepts of the claimed subject matter. In an exemplary embodiment, a method and system for measuring electrophysiological signals from a human brain, the exemplary system comprising a headset unit. In an exemplary embodiment, the headset unit comprising a top base, a circular base, the circular base comprising a peripheral rack and an associated headset rack, a plurality of strips attached to the circular base and the top base, the peripheral rack configured to change respective positions of each respective strip of the plurality of strips, and a plurality of electroencephalograph (EEG) sensors attached to each respective strip of the plurality of strips, each respective EEG sensor of the plurality of EEG sensors configured to adjust its respective height to change position of the respective EEG sensor, each of the plurality of strips configured to change a respective position of each EEG sensor across a length of the respective strip in a second direction. The system may further comprise one or more processors and a storage device that stores a set of instructions that when executed by the one or more processors cause the one or more processors to receive data associated with each EEG sensor, determine whether each EEG sensor is in contact with the subject, and provide instructions to change height of a respective EEG sensor responsive to determining that the respective EEG sensor is not in contact with the subject.

This Summary is provided to introduce a selection of concepts in a simplified form; these concepts are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The novel features which are believed to be characteristic of the present invention, as to its structure, organization, use and method of operation, together with further objectives and advantages thereof, will be better understood from the following drawings in which a presently preferred embodiment of the present disclosure will now be illustrated by way of example. It is expressly understood, however, that the drawings are for the purpose of illustration and description only and are not intended as a definition of the limits of the present disclosure. Embodiments of the present disclosure will now be described by way of example in association with the accompanying drawings in which:

FIG. 1 illustrates a system to measure electrophysiological signals from a subject's brain, consistent with one or more exemplary embodiments of the present disclosure;

FIG. 2 illustrates a schematic view of a headset assembly, consistent with one or more exemplary embodiments of the present disclosure;

FIG. 3 illustrates an exploded view of the headset assembly, consistent with one or more exemplary embodiments of the present disclosure;

FIG. 4 shows an enlarged view of different portions of the headset assembly, consistent with one or more exemplary embodiments of the present disclosure;

FIG. 5 illustrates an enlarged view of different portions of the headset structure, consistent with one or more exemplary embodiments of the present disclosure;

FIG. 6 illustrates enlarged views of different portions of the headset strip, consistent with one or more exemplary embodiments of the present disclosure;

FIG. 7 illustrates a detailed view of a periphery rack, consistent with one or more exemplary embodiments of the present disclosure;

FIG. 8 illustrates a detailed view of a fastener, consistent with one or more exemplary embodiments of the present disclosure;

FIG. 9 illustrates enlarged view of a sensor assembly, consistent with one or more exemplary embodiments of the present disclosure;

FIG. 10 illustrates another exploded view of the sensor assembly, consistent with one or more exemplary embodiments of the present disclosure; and

FIG. 11 illustrates an example computer system in which an embodiment of the present invention, or portions thereof, may be implemented as computer-readable code, consistent with exemplary embodiments of the present disclosure.

### DETAILED DESCRIPTION

The novel features which are believed to be characteristic of the present invention, as to its structure, organization, use and method of operation, together with further objectives and advantages thereof, will be better understood from the following discussion.

As a preliminary matter, some of the figures describe concepts in the context of one or more structural components, variously referred to as functionality, modules, features, elements, etc. The various components shown in the figures can be implemented in any manner, for example, by software, hardware (e.g., discrete logic components, etc.), firmware, and so on, or any combination of these implementations. In one embodiment, the various components may reflect the use of corresponding components in an actual implementation. In other embodiments, any single component illustrated in the figures may be implemented by a number of actual components. The depiction of any two or more separate components in the figures may reflect different functions performed by a single actual component. The figures discussed below provide details regarding exemplary systems that may be used to implement the disclosed functions.

Some concepts are described in form of steps of a process or method. In this form, certain operations are described as being performed in a certain order. Such implementations are exemplary and non-limiting. Certain operations described herein can be grouped together and performed in a single operation, certain operations can be broken apart into plural component operations, and certain operations can be performed in an order that differs from that which is described herein, including a parallel manner of performing the operations. The operations can be implemented by software, hardware, firmware, manual processing, and the like, or any combination of these implementations. As used herein, hardware may include computer systems, discrete logic components, such as application specific integrated circuits (ASICs) and the like, as well as any combinations thereof.

As to terminology, the phrase “configured to” encompasses any way that any kind of functionality can be constructed to perform an identified operation. The functionality can be configured to perform an operation using, for instance, software, hardware, firmware and the like, or any combinations thereof.

As utilized herein, terms “component,” “system,” “client” and the like are intended to refer to a computer-related entity, either hardware, software (e.g., in execution), and/or firmware, or a combination thereof. For example, a component can be a process running on a processor, an object, an executable, a program, a function, a library, a subroutine, and/or a computer or a combination of software and hardware.

By way of illustration, both an application running on a server and the server can be a component. One or more components can reside within a process and a component can be localized on one computer and/or distributed between two or more computers. The term “processor” is generally understood to refer to a hardware component, such as a processing unit of a computer system.

Furthermore, the claimed subject matter may be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any non-transitory computer-readable device, or media.

Non-transitory computer-readable storage media can include but are not limited to magnetic storage devices (e.g., hard disk, floppy disk, and magnetic strips, among others), optical disks (e.g., compact disk (CD), and digital versatile disk (DVD), among others), smart cards, and flash memory devices (e.g., card, stick, and key drive, among others). In contrast, computer-readable media generally (i.e., not necessarily storage media) may additionally include communication media such as transmission media for wireless signals and the like.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

In an exemplary embodiment, an exemplary headset assembly coupled to one or more exemplary processors, provide for a method and system for measurement of electrophysiological signals for brain function diagnostic, brain machine interface, neural prosthetics, and neuroimaging applications. An exemplary system may provide for real time adjustment of the length of dry Electroencephalograph (EEG) measuring electrode sensors according to a scalp size of a subject, positioning of dry EEG measuring electrode sensors on the scalp of the subject, and spatial resolution of the headset according to user requirements, in real time. The exemplary system may utilize one or more processors to receive input from a user (such as a medical professional or a clinical attending), and based on the user input, an exemplary system may adjust the length of EEG electrodes, position the dry EEG measuring electrodes on the headset assembly, and modify spatial resolution of the headset.

In an exemplary embodiment, a headset may provide housing for the placement of duality of replaceable dry EEG measuring electrode sensors on the headset and may provide a mechanism to adjust the height of EEG electrodes sensor in real time according to the size of the scalp of subject for providing direct contact of the dry EEG electrode sensors with the scalp of the subject irrespective of the size of the scalp of subject, grasp the scalp in real time and fixes the electrodes on user defined positions on the headset, in real time. Exemplary systems and methods may provide flexibility to move the dry EEG electrode sensors on the headset assembly by wireless motor controller in real time according to the user input given on processing machine to accurately adjust the placement position of EEG electrode sensors on the headset. In an exemplary embodiment, an exemplary mechanism may allow for adjusting a number of dry EEG electrode sensors on a headset and may provide flexibility on modifying spatial resolution on a single headset according to user requirement. Each EEG electrode sensor on the headset assembly in the disclosure may be connected with an individual insulated cable (or any other method or mechanism) to carry the measured EEG signals from a person's brain to the processing machine. The processing machine may display the EEG measurements of each EEG electrode sensor on it and may also display the current positioning of each dry EEG electrode sensor on the headset. In an exemplary embodiment, such a configuration may provide flexibility to a user to modify the positioning of EEG electrodes according to the requirement of the EEG signal measurements, in real time.

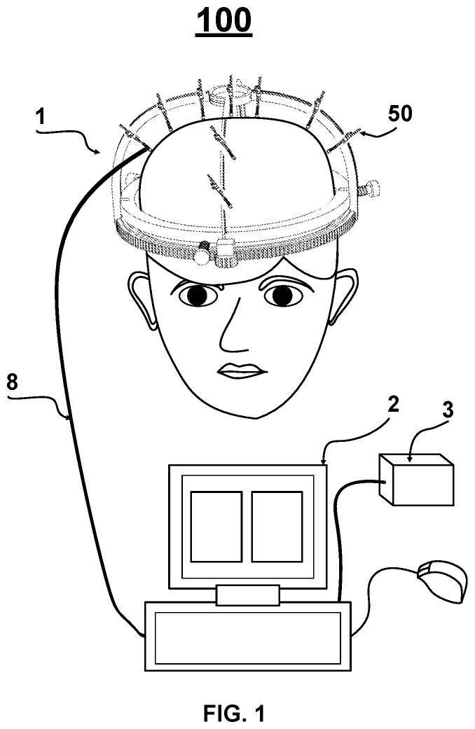

FIG. 1 illustrates a system to measure electrophysiological signals from a subject's brain, consistent with one or more exemplary embodiments of the present disclosure. Specifically, exemplary system 100 may comprise of a headset assembly 1, a processing machine 2 and a wireless motor controller 3. The headset assembly 1 may contain a plurality of EEG electrode sensors 50 to measure EEG signals from the desired locations on the scalp of a subject. In an exemplary embodiment, each of the EEG measurements captured from each respective EEG electrode sensor 50 may be transmitted to a computing system 2 through an insulated cable 8 (or any other method or mechanism) to transfer EEG signals from respective EEG electrode sensors to the computing system. In an exemplary embodiment, computing system 2 may display the EEG measurements on an exemplary monitor or display.

In an exemplary embodiment, wireless motor controller 3 may control movements, real-time positions and heights of the EEG electrode sensors 50 on the headset assembly 1, based on user input, instructions, or requirements. A display of computing system 2 may display respective positions of EEG electrode sensors 50 on headset assembly 1, in real time.

In an exemplary embodiment, processing machine 2 may be connected with each individual EEG electrode sensor 50 of the EEG headset assembly 1, utilizing a conducting cable 8 (or any other method or mechanism). In an exemplary embodiment, processing machine 2 may also be connected with the wireless motor controller 3. The EEG data display module 4 may be utilized to show the EEG data of each EEG electrode sensor 50 of the headset assembly 1. The real time sensor position module 5 may be utilized to display the real time position of each EEG electrode sensor 50 on the headset assembly 1. The input device may be utilized to receive input from a user to adjust respective heights of the EEG electrode sensors 50 and their positions on the headset assembly 1. The wireless controller 3 may get information of the EEG electrode sensor 50 position provided by the user in real time and send the wireless signals to the headset strip rotating motor 26, sensor assembly headset moving motor 58 and sensor assembly height adjustable motor 60 to rotate to adjust the height of each EEG electrode sensor 50 and their position on the headset assembly 1.

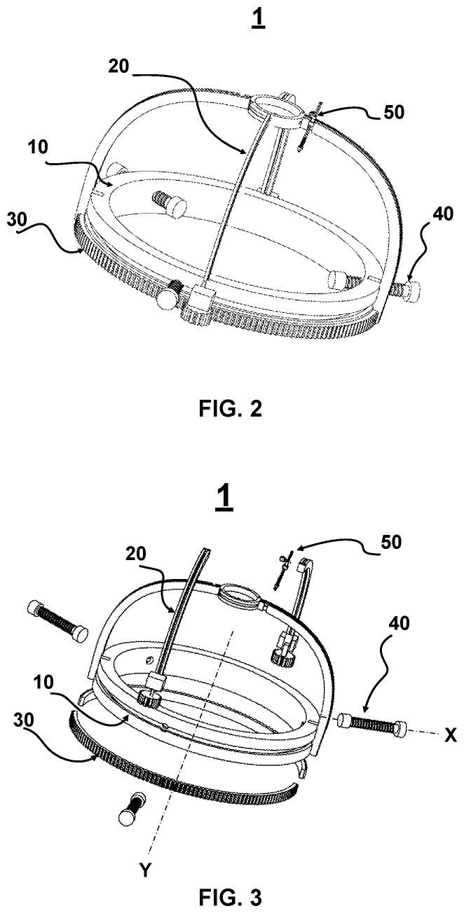

FIG. 2 illustrates a schematic view of a headset assembly, consistent with one or more exemplary embodiments of the present disclosure. In an exemplary embodiment, headset assembly 1 may comprise of headset structure 10, duality of headset strips 20, periphery rack 30, multiple fasteners 40, and a plurality of EEG electrode sensors, represented by EEG electrode sensor 50. In an exemplary embodiment, headset structure 10 may provide the housing to assemble multiple number of headset strips 20 on it, assemble peripheral rack 30 on it, and may provide housing to fix head set assembly 1 on a scalp of a potential subject using multiple number of fasteners 40. In FIG. 2, the headset strips 20 in the headset assembly 1 may be changed, replaced, and positioned at different locations on periphery rack 30. The position of headset strip 20 may be adjusted by the rotation of headset strip rotating gear 27 on periphery rack 30 by the rotation of head strip rotating motor 26. Furthermore, on each headset strip 20, a plurality of EEG electrode sensors 50 may be placed, moved and fixed at different positions on the headset strip 20.

FIG. 3 illustrates an exploded view of the headset assembly, consistent with one or more exemplary embodiments of the present disclosure. Specifically, an exploded view of the headset assembly 1 is illustrated. In an exemplary embodiment, in headset assembly 1, the headset structure 10 may be utilized to provide housing to place a plurality of headset strips 20. In FIG. 3, multiple replaceable headset strips 20 may be placed on the headset assembly 1 and may change their position around the outer surface 301 of the periphery rack 30 of the headset assembly 1 by movement of headset strip rotating gear 27 on periphery rack 30 on the peripheral rack 30. In FIG. 3, each headset strip 20, may provide the housing to place a plurality of EEG electrode sensors 50 and may allow EEG electrode sensors 50 to be moved and to be fixed at different positions on the headset strips 20.

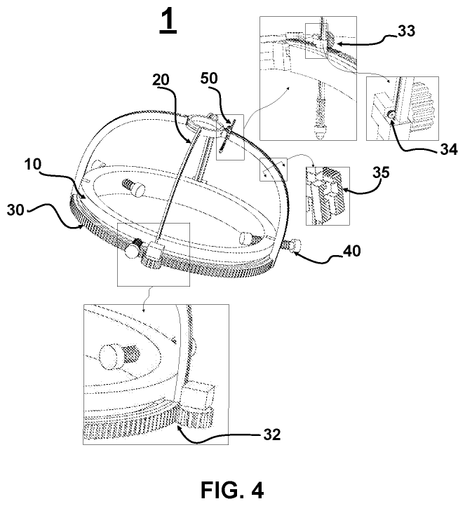

FIG. 4 shows an enlarged view of different portions associated with a moving mechanism of the headset assembly, consistent with one or more exemplary embodiments of the present disclosure. Specifically, an enlarged view of the moving mechanism 32 of headset strips 20 on the outer surface 301 of the peripheral rack 30 is provided, moving mechanism 33 of EEG electrode sensors 50 on the outer surface 121 of the headset strips 20, moving mechanism 34 of EEG electrode sensors 50 to adjust height of EEG electrode sensors 50 according to the size of the scalp of subject and cross section enlarge view 35 of the headset strips 20 is shown.

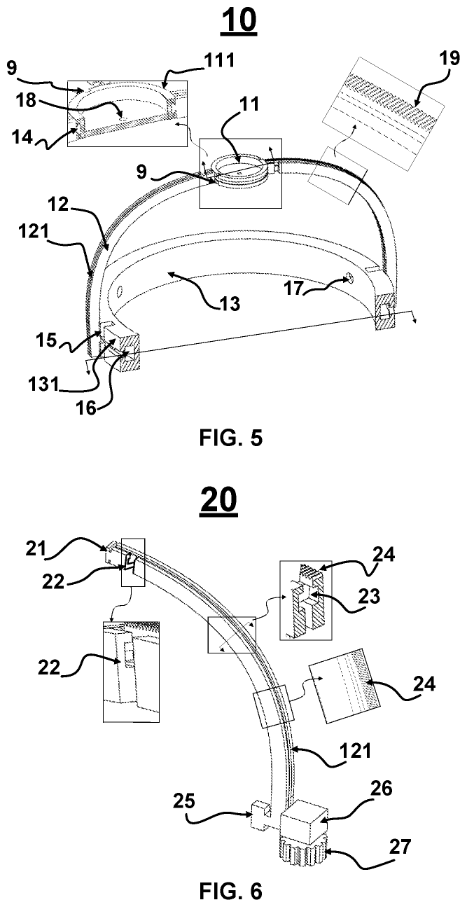

FIG. 5 illustrates an enlarged view of different portions of the headset structure, consistent with one or more exemplary embodiments of the present disclosure. Headset structure 10 may comprise of top base 11, fixed headset strips 12 and headset frame 13. The top base 11, may be part of a headset structure 10 that may be utilized to provide support to insert the top side 21 of the headset strips 20 and may also aid in fixing headset strips 12 on the outer edges 111 of the top base 11. In an exemplary embodiment, a T-slot 14 may be provided on outer edges 111 of the top base 11 which may aid in constraining movement of headset strips 20 in the headset assembly 1. Additionally, in an exemplary embodiment, there may be provided a top base opening 9 connected with the T-slot 14 in the top base 11 from where headset strips 20 may be inserted and removed from the headset assembly 1. The fixed headset strips 12 on the headset structure 10 may utilized to join top base 11 and headset frame 13 to each other. Furthermore, fixed headset strips 12 and the headset strips 20 have same cross section 23. There is strip opening 22 provided in fixed headset strips 12 and the headset strips 20. The strip opening 22 provided in headset strips 12 and the headset strips 20 aids EEG electrode sensors 50 to insert and removed from the fixed headset strips 12 and the headset strips 20. In an exemplary embodiment a moving mechanism 33 of EEG electrode sensors 50 may be provided on the outer surface 121 of the headset strips 20. In the moving mechanism 33, there may be gear teeth 19 on the outer surface 121 of the fixed headset strips 12 and the headset strips 20 which are utilized to move the EEG electrode sensors 50 along the outer surface 121 of the fixed headset strips 12 and the headset strips 20. The teeth 591 of the sensor assembly headset moving gear 59 mesh with the gear teeth 24 on the outer surface 121 of the headset strip 20. The rotation of the sensor assembly headset moving gear 59 by the sensor assembly headset moving motor 58, causes movement of the EEG electrode sensors 50 on the outer surface 121 of the headset strips 20. The headset frame 13 is provided in the headset structure 10 to support to insert the bottom side 25 of the headset strips 20 and also fixes two number of fixed headset strips 12 on the outer surface 121 of the headset frame 13. There is provided a T-slot 16 in the outside surface 131 of the head set frame 13 headset frame 13 which can constraint the movement of the headset strips 20 in the headset assembly 1 and there is provided a head set frame opening 15 connected with the T-slot 16 on the outside surface 131 of the periphery of the head set frame 13 from where headset strips 20 can be inserted and removed from the headset assembly 1. Furthermore, there are provided fastener holes 17 on the headset frame 13 which are utilized to insert the fasteners 40 to fix the headset assembly 1 with the scalp of the subject.

FIG. 6 illustrates enlarges views of different portions of the headset strip 20 consistent with one or more exemplary embodiments of the present disclosure. In an exemplary embodiment, head set strip 20 may comprises of top side 21, side opening 22, T-slot 23, gear teeth 24 on the outer surface 121 of the headset strip 20, bottom surface 25, headset strip rotating motor 26, and a headset strip rotating gear 27. The top side 21 of the headset strip 20 may be utilized to insert the headset strip 20 in the top base opening 9 of the T-slot 14 which may be provided on the top base 11 of the headset structure 10 of the headset assembly 1. In an exemplary embodiment, the combination of tope base opening 9 and T-slot 14 may aid in constraining the headset strips 20 to move in the T-slot 14 which may be provided on the top base 11 of the headset structure 10 of the headset assembly 1. The side opening 22 may be provided in the headset strip 20 and connected with the T-slot 23, may be utilized to insert the EEG electrode sensors 50 into the headset strip 20. The T-slot 23 may be provided in the headset strip 20 to aid in constraining movement of the EEG electrode sensors 50 to move along the outer surface 121 of the headset strip 20. In an exemplary embodiment, periphery of the head strip 20 may refers to the circular outer surface 121 of the headset strip 20. The gear teeth 24 on the outer surface 121 of the headset strip 20 may be utilized to move EEG electrode sensors 50 along outer surface 121 of the headset strip 20. The bottom surface 25 of the headset strip 20 may be provided to inset the headset strip 20 in the opening 15 of the T-slot 16 which may be provided on the outside surface 131 of the headset frame 13 of the headset assembly 1. The headset strip rotating motor 26 may be fixed with the headset strip 20 to drive/rotate the headset strip rotating gear 27. The headset strip rotating gear 27 may mesh with the gear teeth 31 on the outer surface 302 of the rack 30 of the headset assembly 1 and may allow the headset strips 20 to move along the outer surface 302 of the periphery rack 30 of the headset assembly 1 with the controlled rotations of the headset strip rotating motor 26.

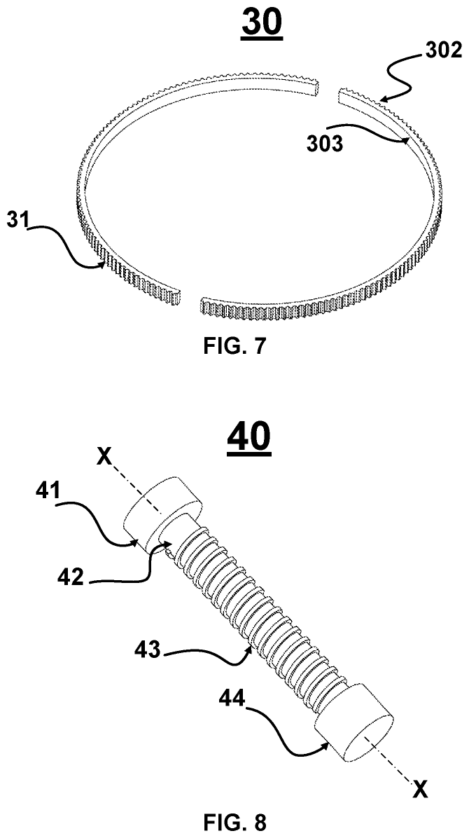

FIG. 7 illustrates a detailed of a periphery rack 30, consistent with one or more exemplary embodiments of the present disclosure. In an exemplary embodiment, periphery rack 30 may contain gear teeth 31 on its outer circular surface 302 and it may be assembled around the outer side surface 131 of headset frame 13 by making contact of the inside surface 303 of the periphery rack 30 with the outside surface 131 of the headset frame 13. Accordingly, periphery rack 30 may provide a mechanism 32 to assemble headset strip rotating gear 27 by meshing headset strip rotating gear 27 with the gear teeth 31 of the periphery rack 30 of the headset assembly 1. Such a configuration, may allow headgear strips 20 to move around the outer surface 302 of the rack 30 by the rotation of headset strip rotating gear 27 of the headset assembly 1.

FIG. 8 illustrates a detailed view of a fastener, consistent with one or more exemplary embodiments of the present disclosure.

In an exemplary embodiment, fastener 40 may comprise of fastener head 41, fastener shank 42, square threads 43, on the outer circular surface of the shank 42 and fastener scalp seat 40. The fastener head 41, fastener shank and fastener seat are joined together on the same axis X. The fastener 40 may be assembled on the fastener holes 17 on the headset frame 13 of the headset assembly 1. The fastener head 41 may be utilized to rotate the fastener 40 in the fastener holes 17. The fastener shank 42 may connect the fastener head 41 with the fastener scalp seat 44. In an exemplary embodiment, there are square threads 43 (or any other type of threads such as unified threads, metric, ACME, Buttress threads etc) provided on the body of fastener shank 42. In another exemplary embodiment, other threads with similar functionality as square threads 43 may be utilized, such as unified threads, metric, ACME, Buttress threads etc. Rotation of fastener head 41 by a user, may allow inside and outside movement of the fastener 40 from the headset frame 13 of the headset assembly 1. In an exemplary embodiment, inside and outside movement may refer to the movement of the scalp seat 44 of the fastener 40 towards the axis Y of the headset frame 13, while outside movement may refer to the movement of the scalp seat 44 of the fastener 40 away from the axis Y of the headset frame 13.

In an exemplary embodiment, the fastener scalp seat 44 may be made of a soft material. The fastener is head 41 may be rotated to rotate the fastener 40 in the holes 17 and causes inside or outside movement of the scalp seat 44 of the fastener 40. The inside movement of scalp seat 44 of the fastener 40 may cause direct contact of the fastener seat 44 with the scalp of the subject. While the outsize movement of the fastener seat 44 causes to remove the direct contact of the fastener seat 44 with the scalp of the subject. A plurality of the fasteners 40 may be utilized in the headset assembly 1 to grasp different size of the scalp of the subject.

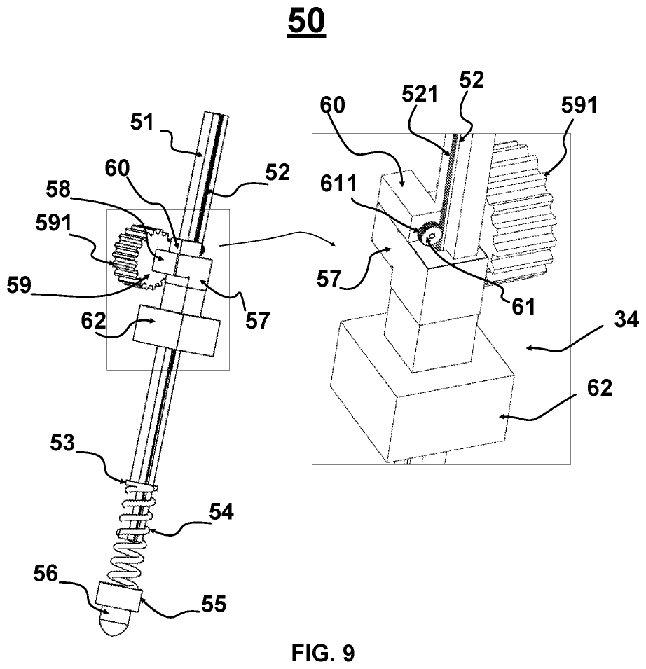

FIG. 9 illustrates enlarged view of a sensor assembly, consistent with one or more exemplary embodiments of the present disclosure. In an exemplary embodiment, the sensor assembly 50 may comprise of sensor rack 51, sensor rack gear teeth 52, spring seat 53, spring 54, sensor seat 55, dry EEG sensor 56, motor bracket 57, sensor assembly headset moving motor 58, sensor assembly headset moving gear 59, sensor assembly height adjustable motor 60, sensor assembly height adjustable pinion 61, and sensor assembly base 62. The sensor rack 51 may be utilized to adjust the sensor assembly 50 to a desired length based on the size of the scalp of the subject for making direct contact of the fastener seat 44 with the scalp of the subject. The height of the sensor rack 51 may give flexibility to the sensor assembly 50 to adjust a height of the sensor assembly 50 from the surface of the scalp of a subject, thereby, it may allow a headset assembly headset assembly 1 to become wearable to different size of the scalp of subject. In an exemplary embodiment, sensor assembly 50 may contain moving mechanism 34 of EEG electrode sensors 50 to adjust height of EEG electrode sensors 50 according to the size of the scalp of subject. Sensor rack gear 52 in the moving mechanism 34 may be provided on the sensor rack 51 to mesh the teeth 611 of sensor assembly height adjustable pinion 61 with the teeth of the sensor rack gear 521. In an exemplary embodiment, sensor seat 53 may be provided on the sensor rack 51 which may aid in constraining movement of spring 54 on the sensor rack 51. In an exemplary embodiment, spring 54 may be provided in the sensor assembly 50 to (or to make other minor changes), in the height of the dry EEG sensor 56 from the scalp of the subject and gives the flexibility to the dry EEG sensor 56 to adjust its minor height from the scalp of the subject if there are irregular surface on the scalp of the subject. Spring seat 55 is provided in the sensor assembly 50 to fix the dry EEG electrode 56 and the spring 54 in the sensor assembly 50. Motor bracket 57 is provided in the sensor assembly 50 to give housing to the sensor assembly headset moving motor 58 and sensor assembly height adjustable motor 60. The sensor assembly headset moving motor 58 is utilized to rotate the sensor assembly headset moving gear 59 which is meshed with the gear teeth on the top surface 24 of the headset strip 20 and gear teeth 19 on the top surface of the fixed headset strips 12 and allows the movement of the sensor assembly 50 around the periphery of the fixed headset strips 12 and the headset strip 20. The sensor assembly height adjustable motor 60 is provided in the sensor assembly 50 to give rotation to the sensor assembly height adjustable pinion 61 which is meshed with the sensor rack gear teeth 52 provided on the surface of the sensor rack 51 and allows the vertical movement of the sensor rack 51. The vertical movement of the sensor rack 51 allows the direct contact of the dry EEG sensor 56 with a scalp of the subject, therefore, it may allow for adjusting the height of the EEG electrode assembly 50. In an exemplary embodiment, the sensor assembly base 62 may be provided in the sensor assembly 50 which may allow the sensor assembly 50 to insert the electrode assembly 50 into the side opening 22 of the headset strip 20 and may allow sensor assembly 50 to move in the T-slot 23 of the headset strip 20.

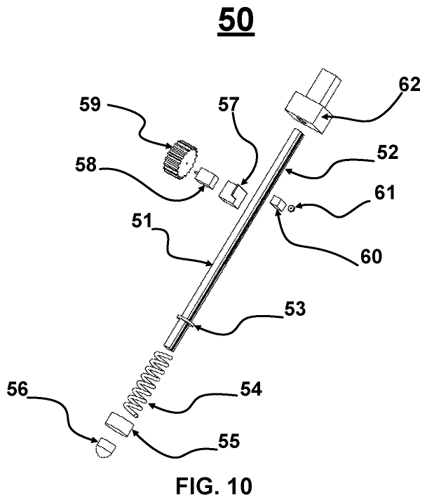

FIG. 10 illustrates an exploded view of the sensor assembly, consistent with one or more exemplary embodiments of the present disclosure. In the exemplary scenario disclosed, sensor rack 51 may be inserted into the sensor base 62 and the motor bracket 57 in the sensor assembly 50 to allow other parts of the sensor assembly 50 to assemble.

In an exemplary embodiment, exemplary systems and methods may utilize EEG data captured by exemplary EEG sensors and data related to positions of the sensors to generate a two dimensional or a three dimensional representation of a head of a subject. In an exemplary embodiment, the generated two or three dimensional representation of a head of a subject may be displayed. In an exemplary embodiment, one or more exemplary embodiments, one or more processors, may utilize the shape of the head of a subject, positional data from an exemplary headset, regarding position of the EEG sensors, and may move the sensors so that all of the EEG sensors are in contact with a subjects head to ensure that data is captured from all respective EEG sensors.

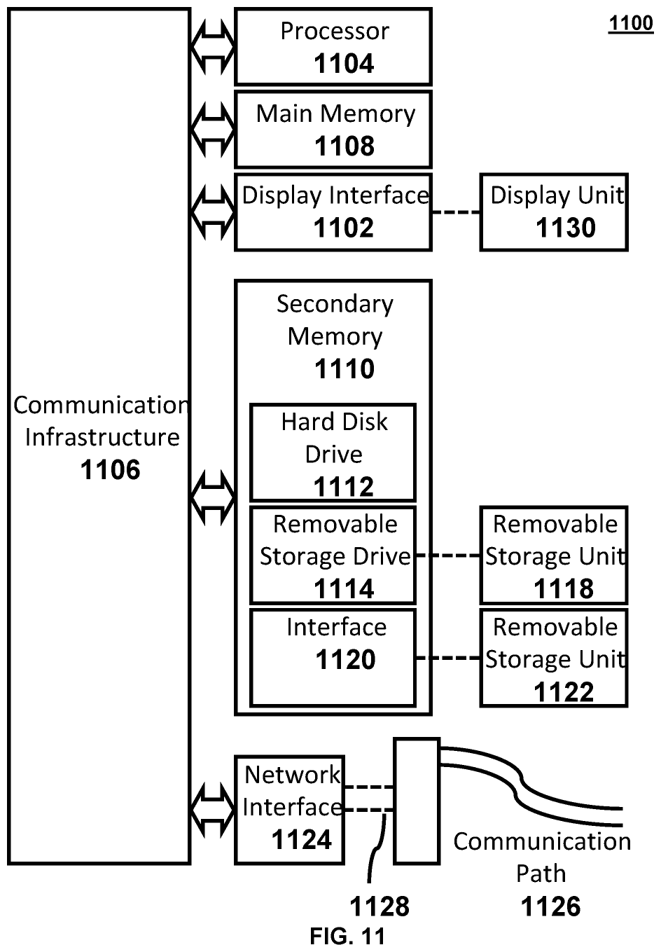

FIG. 11 illustrates an example computer system 1100 in which an embodiment of the present invention, or portions thereof, may be implemented as computer-readable code, consistent with exemplary embodiments of the present disclosure. For example, computing system 2 may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components utilized with respect to any methods described in context of computer operations related to the functioning of systems disclosed in FIGS. 1-10.

Digital camera attached to a port on the light microscope and to a computer system via USB/Ethernet cable or other means. Application that allows operator to take a picture when oocyte is ready to be captured. Application that performs analysis and displays prediction result.

If programmable logic is used, such logic may execute on a commercially available processing platform or a special purpose device. One of ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 1100. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 1104 may be a special purpose or a general-purpose processor device. As will be appreciated by persons skilled in the relevant art, processor device 1104 may also be a single processor in a multi-core/multiprocessor system, such system operating alone, or in a cluster of computing devices operating in a cluster or server farm. Processor device 1104 is connected to a communication infrastructure 1106, for example, a bus, message queue, network, or multi-core message-passing scheme.

Computer system 1100 also includes a main memory 1108, for example, random access memory (RAM), and may also include a secondary memory 1110. Secondary memory 1110 may include, for example, a hard disk drive 1112, removable storage drive 1114. Removable storage drive 1114 may comprise a floppy disk drive, a magnetic tape drive, an optical disk drive, a flash memory, or the like. The removable storage drive 1114 reads from and/or writes to a removable storage unit 1118 in a well-known manner. Removable storage unit 1118 may comprise a floppy disk, magnetic tape, optical disk, etc., which is read by and written to by removable storage drive 1114. As will be appreciated by persons skilled in the relevant art, removable storage unit 1118 includes a computer usable storage medium having stored therein computer software and/or data.

In alternative implementations, secondary memory 1110 may include other similar means for allowing computer programs or other instructions to be loaded into computer system 1100. Such means may include, for example, a removable storage unit 1122 and an interface 1120. Examples of such means may include a program cartridge and cartridge interface (such as that found in video game devices), a removable memory chip (such as an EPROM, or PROM) and associated socket, and other removable storage units 1122 and interfaces 1120 which allow software and data to be transferred from the removable storage unit 1122 to computer system 1100.

Computer system 1100 may also include a communications interface 1124. Communications interface 1124 allows software and data to be transferred between computer system 1100 and external devices. Communications interface 1124 may include a modem, a network interface (such as an Ethernet card), a communications port, a PCMCIA slot and card, or the like. Software and data transferred via communications interface 1124 may be in the form of signals, which may be electronic, electromagnetic, optical, or other signals capable of being received by communications interface 1124. These signals may be provided to communications interface 1124 via a communications path 1126. Communications path 1126 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 1118, removable storage unit 1122, and a hard disk installed in hard disk drive 1112. Computer program medium and computer usable medium may also refer to memories, such as main memory 1108 and secondary memory 1110, which may be memory semiconductors (e.g. DRAMs, etc.).

Computer programs (also called computer control logic) are stored in main memory 1108 and/or secondary memory 1110. Computer programs may also be received via communications interface 1124. Such computer programs, when executed, enable computer system 1100 to implement the present invention as discussed herein. In particular, the computer programs, when executed, enable processor device 1104 to implement the processes of the present invention, such as capturing data, generating image data, etc. Accordingly, such computer programs represent controllers of the computer system 1100. Where the invention is implemented using software, the software may be stored in a computer program product and loaded into computer system 1100 using removable storage drive 1114, interface 1120, and hard disk drive 1112, or communications interface 1124.

Embodiments of the invention also may be directed to computer program products comprising software stored on any computer useable medium. Such software, when executed in one or more data processing device, causes a data processing device(s) to operate as described herein. An embodiment of the invention employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

Other modifications and alterations may be used in the design and manufacture of the apparatus of the present invention without departing from the spirit and scope of the accompanying claims.

Throughout this specification and the claims which follow, unless the context requires otherwise, the word “comprise”, and variations such as “comprises” or “comprising”, will be understood to imply the inclusion of a stated integer or step or group of integers or steps but not to the exclusion of any other integer or step or group of integers or steps.

Moreover, the word “substantially” when used with an adjective or adverb is intended to enhance the scope of the particular characteristic; e.g., substantially planar is intended to mean planar, nearly planar and/or exhibiting characteristics associated with a planar element. Further use of relative terms such as “vertical”, “horizontal”, “up”, “down”, and “side-to-side” are used in a relative sense to the normal orientation of the apparatus.

What is claimed:

1. A system for measuring electrophysiological signals from a human brain, the system comprising:
a headset unit, comprising:
a top base;
a circular base, the circular base comprising a peripheral rack and an associated headset rack;
a plurality of strips attached to the circular base and the top base, the peripheral rack configured to change respective positions of each respective strip of the plurality of strips;
a plurality of electroencephalograph (EEG) sensors attached to each respective strip of the plurality of strips, each respective EEG sensor of the plurality of EEG sensors configured to adjust its respective height to change position of the respective EEG sensor, each of the plurality of strips configured to change a respective position of each EEG sensor across a length of the respective strip in a second direction;
one or more processors; and
a storage device that stores a set of instructions that when executed by the one or more processors cause the one or more processors to:
receive data associated with each EEG sensor;
determine whether each EEG sensor is in contact with the subject; and
provide instructions to change height of a respective EEG sensor responsive to determining that the respective EEG sensor is not in contact with the subject.

2. The system for measuring electrophysiological signals from a human brain of claim 1, wherein the headset unit further comprising:
a plurality of fasteners attached to the circular base, the plurality of fasteners configured to attach the headset unit to a subject's head.

3. The system for measuring electrophysiological signals from a human brain of claim 1, wherein the peripheral rack further comprising:
a rotating motor associated with a rotating gear for each respective strip of the plurality of strips, wherein the peripheral rack comprising gear teeth, the rotating gear configured to change position of the respective strip by interacting with the gear teeth.

4. The system for measuring electrophysiological signals from a human brain of claim 3, wherein each EEG sensor configured to change its respective height utilizing a sensor rack gear teeth, a sensor assembly height adjustable motor, and a sensor assembly height adjustable pinion.

5. The system for measuring electrophysiological signals from a human brain of claim 4, wherein each EEG sensor configured to change its respective height utilizing the sensor assembly height adjustable motor by rotating a respective amount corresponding to a required height change.

6. The system for measuring electrophysiological signals from a human brain of claim 1, wherein the second direction perpendicular to a direction in the change in the position of the respective EEG sensor in a flat state for the respective strip.
